(19) United States
(12) Patent Application Publication
(10) Pub. No.: US 2015/0184193 A1
(43) Pub. Date: Jul. 2, 2015

(54) TRAIT STACKING STRATEGY FOR CORN INTROGRESSION

(71) Applicant: Dow AgroSciences LLC, Indianapolis, IN (US)

(72) Inventors: Janelle MEYER, Zionsville, IN (US); James W. BING, Zionsville, IN (US)

(21) Appl. No.: 14/581,365

(22) Filed: Dec. 23, 2014

Related U.S. Application Data
(60) Provisional application No. 61/921,681, filed on Dec. 30, 2013.

(51) Int. Cl. C12N 15/82; A01H 1/04; A01H 1/02

(52) U.S. Cl. CPC C12N 15/8286; A01H 1/02; C12N 15/8274; C12N 15/8273; C12N 15/8243; A01H 1/04

(57) ABSTRACT

A method is provided to decrease the time required to introgress three or more desired traits from donor plant lines into an elite plant background. The method comprises crossing two donor plants, wherein the donor plants have each been backcrossed to have a high recurrent parent percentage and share one desired locus to be introgressed into the elite plant background.

### CROSS-REFERENCE TO RELATED APPLICATIONS

This application claims the benefit under 35 USC §119(e) of U.S. Provisional Application Ser. No. 61/921,681, filed on Dec. 30, 2013, the entire disclosure of which is incorporated herein by reference.

### BACKGROUND

Plant breeding is the art and science of producing plants that have new combinations of desired characteristics. Plant breeding can be accomplished through many different techniques ranging from simply selecting plants with desirable characteristics for propagation, to more complex molecular techniques. Traditional plant breeding methods still have a place in the development of plant varieties, even with all of the advances in molecular biology and the ability to produce trangenic plants. One important use of traditional plant breeding methods involves the use of backcross procedures to move a transgene from a good tissue culture variety that was used in transformation to an elite experimental line or variety. For many crops, once the transgene is in the crop species, crossing is more efficient than transforming the elite line via recomnbinant technology because most transformation protocols are optimized for a specific (often poorly adapted and lower yielding) laboratory line. Many elite lines (which are high yielding) are not amenable to transformation. Hence genetic engineers typically transform lab lines and breeders backcross the transgene from the lab line into the elite line.

However, standard backcross breeding methodologies are time consuming. Introgressing three genes into elite inbred germplasm typically requires four years from start to produce seed. Accordingly, there is a need for new backcrossing breeding methods that can reduce this timeframe. The present disclosure provides a methodology to eliminate one or more generations of backcrossing and allows for larger and faster parent seed increases when three or more transgenic events are to be introgressed into a recurrent parent.

### SUMMARY

In accordance with one embodiment an improved backcross breeding methodology is provided to reduce the time required to introgress three or more stacked traits or nucleic acids segments into an elite inbred plant line. In one embodiment the method comprises crossing two donor plants, wherein the two donor plants each comprise one of the three desired stacked trangenic events in common and each donor has been backcrossed with the same recurrent plant to generate backcross donor lines that have a high recurrent parent percentage and carry the genes of interest from the donor. The two backcross donor lines (each with a different set of genes from the donors) are then crossed to produce progeny that comprise the three trangenic events originally present in the two donor parent lines and has a recurrent parent percentage of at least 94%.

In one embodiment the method for introgressing three or more transgenic events from two donor lines into a single recurrent plant germplasm comprises crossing a first donor/recurrent plant with a second donor/recurrent plant, wherein the first donor/recurrent plant comprises a first transgenic event and a second transgenic event, and the second donor/recurrent plant comprises the second transgenic event, and a third transgenic event, with both donor/recurrent plants having greater than 80% of a recurrent plant genome. After crossing the two donor/recurrent plants, product plant progeny are examined to identify and select those that comprise said first, second and third transgenic events. In one embodiment the first donor/recurrent plant and the second donor/recurrent plant are generated as parallel plant lines wherein each of the first donor plant and second donor plant is crossed with the same recurrent plant to generate first and second progeny, respectively, wherein said first donor plant comprises the first transgenic event and the second transgenic event and the second donor plant comprises the second transgenic event and the third transgenic event. One or more backcross generations are then generated by crossing the first plant progeny, and the second plant progeny, with the recurrent parent plants to provide first and second backcross plants, respectively. The first and second backcross plants are then backcrossed with said recurrent plants to produce a first donor/recurrent plant (comprising said first transgenic event, the second transgenic event and having greater than 80% of the recurrent plant genome), and a second donor/recurrent plant (comprising said second transgenic event, said third transgenic event and having greater than 80% of the recurrent plant genome), respectively. In one embodiment, in at least one of the crosses conducted with the recurrent plant within each donor conversion, the recurrent parent plant is a female plant.

In one embodiment a method for introgressing three or more transgenic events into a recurrent plant germplasm is provided wherein the method comprises
a) providing a first donor plant, comprising a first stack of at least two transgenic events;
b) crossing the first donor plant with a selected recurrent parent plant to produce a first F1 progeny plant that comprises the first stack of transgenic events;
c) performing a first breeding backcross of the first F1 progeny plant with the recurrent parent plant, and selecting a first breeding backcross progeny plant comprising the first stack of transgenic events
d) backcrossing the selected first breeding backcross progeny with the recurrent parent plant one or more times in succession to produce a BC2 or higher first backcross progeny plant comprising the first stack of transgenic events;
e) selecting a second donor plant comprising a second stack of at least two transgenic events, wherein at least one of the transgenic events of the second stack is also present in the first stack of transgenic events;
f) crossing the second donor plant and the selected recurrent parent plant to produce a second F1 progeny plant that comprises the second stack of transgenic events;
g) performing a second breeding backcross of the second F1 progeny plant to the recurrent parent plant, and selecting a second breeding backcross progeny plant comprising the second stack of transgenic events;
h) backcrossing the selected second breeding backcross progeny to the recurrent parent plant one or more times in succession to produce an BC2 or higher second backcross progeny plant comprising the second stack of transgenic events;
i) crossing the BC2 or higher first backcross progeny with the BC2 or higher second backcross progeny to produce a third progeny plant comprising the unique three transgenic events from the first and second stacks of transgenic events.

### BRIEF DESCRIPTION OF THE DRAWINGS

FIG. 1 Schematic drawing of the trait conversion strategy for stacking Mon 88017 and Mon89034 along with TC1507 into an elite germplasm and recover 98% recurrent parent genome.

FIG. 2 provides a flowchart of the marker assisted selection scheme to be used for BC2 and BC3 progeny of the parallel conversions for Mon17 (Mon88017::TC1507) and Mon89 (Mon88017::Mon89034).

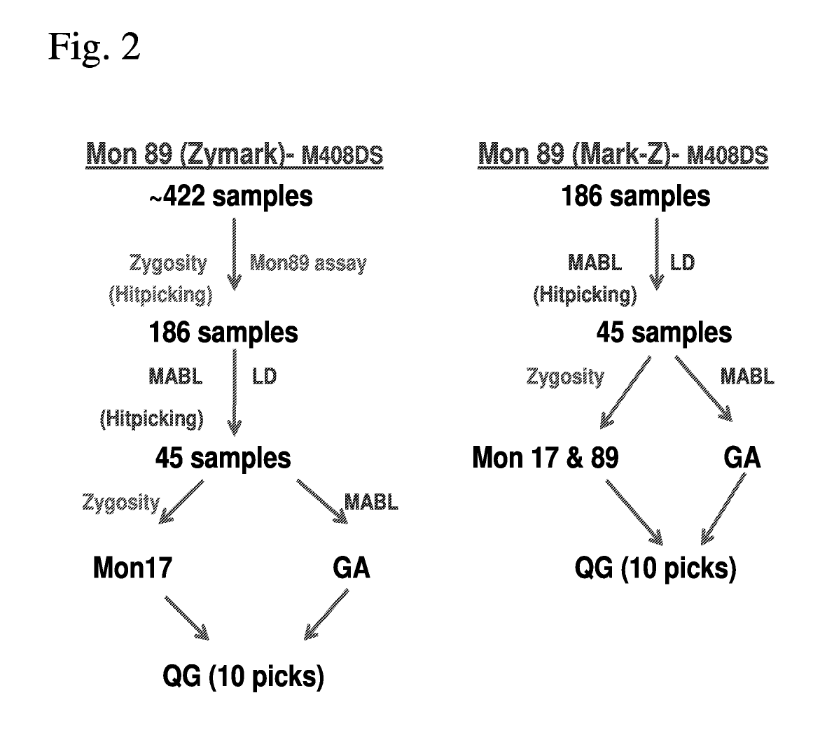

FIG. 3 provides a flowchart of the marker assisted selection scheme for BC2.

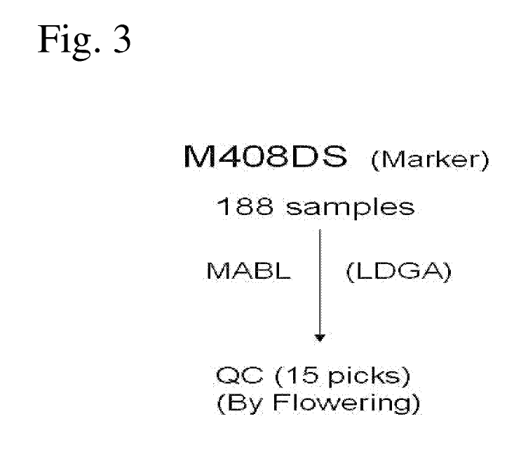

FIG. 4 provides a flowchart of the marker assisted selection scheme for BC3.

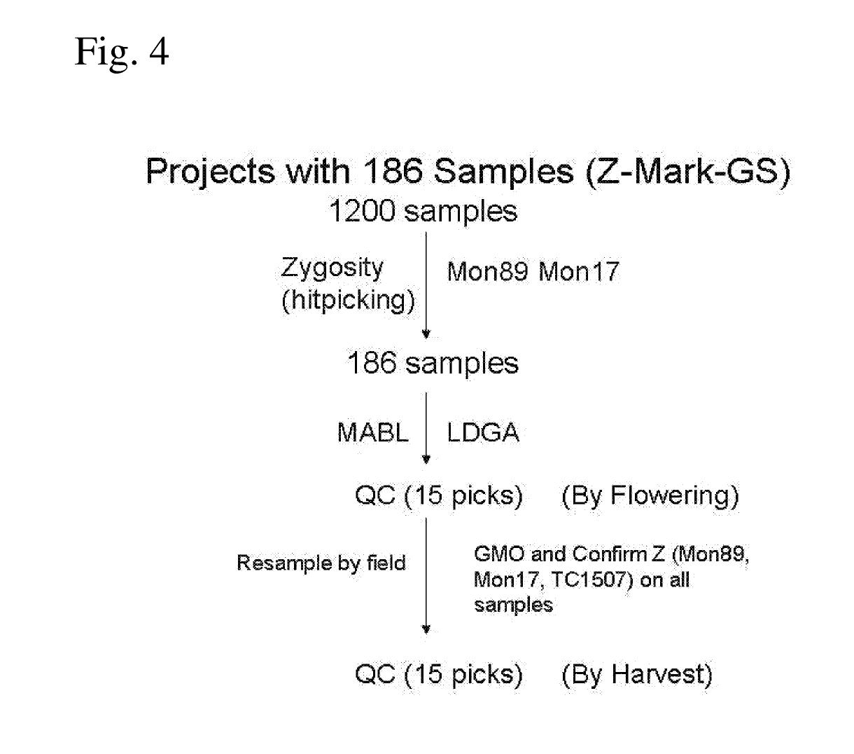

FIG. 5 provides a flowchart of the marker assisted selection scheme for the Stacking Generation.

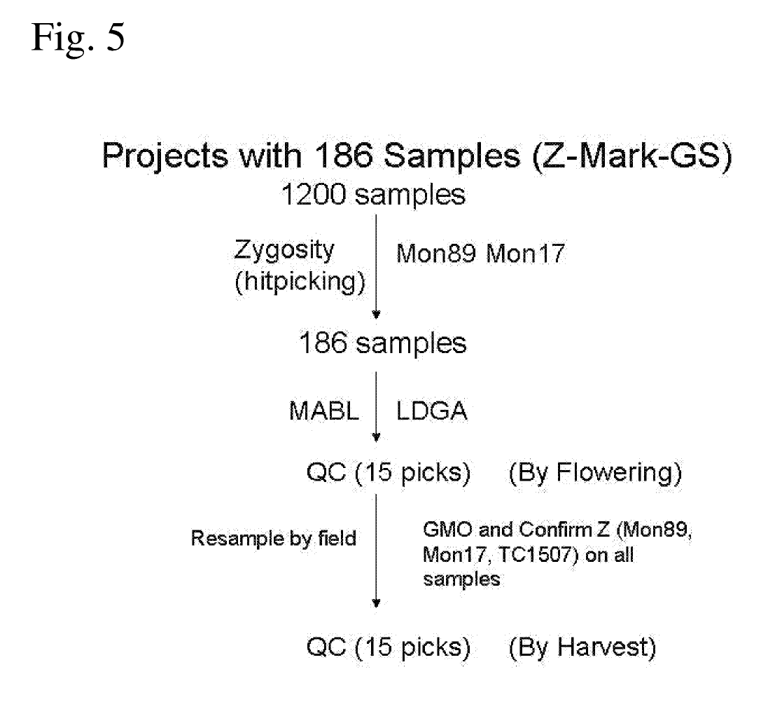

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein, “endogenous sequence” defines the native form of a polynucleotide, gene or polypeptide in its natural location in the organism or in the genome of an organism.

The term “isolated” as used herein means having been removed from its natural environment.

The term “purified,” as used herein defines an isolation of a molecule or compound in a form that is substantially free of contaminants normally associated with the molecule or compound in a native or natural environment and means having been increased in purity as a result of being separated from other components of the original composition. The term “purified nucleic acid” is used herein to describe a nucleic acid sequence which has been separated from other compounds including, but not limited to polypeptides, lipids and carbohydrates.

The term “exogenous DNA sequence” as used herein is any nucleic acid sequence that has been removed from its native location and inserted into a new location altering the sequences that flank the nucleic acid sequence that has been moved. For example, an exogenous DNA sequence may comprise a sequence from another species.

As used herein a “modified endogenous sequence” is an alteration in an endogenous nucleic acid sequence and includes deletions, insertions, substitutions and rearrangement of endogenous genomic sequences, including the insertion of exogenous DNA sequences.

As used herein the term “transgenic event” is intended to designate a locus encoding for one or more desired trait. The transgenic event may comprises a modified endogenous sequence representing a single change or may include a series of modified endogenous sequences that segregate together, including for example, one or more tightly linked exogenous sequences that may contain one or more transgenes.

As used herein, the terms “introgression”, “introgressed” and “introgressing” refer to both a natural and artificial process, and the resulting events, whereby traits, genes or DNA sequences of one species, variety or cultivar are moved into the genome of another species, variety or cultivar, by crossing those species. The process may optionally be completed by backcrossing to the recurrent parent. Examples of introgression include entry or introduction of a gene, a transgene, a regulatory element, a marker, a trait, a trait locus, or a chromosomal segment from the genome of one plant into the genome of another plant.

“Locus” (plural loci) refers to the specific location of a gene or DNA sequence in a genome. A locus may confer a specific trait and may be present in the nuclear, chloroplast or mitochondrial DNA.

As used herein the term “recurrent parent” or “recurrent plant” describes an elite line that is the recipient plant line in a cross and which will be used as the parent line for successive backcrosses to produce the final desired line.

The term “crossing” as used herein refers to the fertilization of female plants (or gametes) by male plants (or gametes). The term “gamete” refers to the haploid reproductive cell (egg or pollen) produced in plants by meiosis from a gametophyte and involved in sexual reproduction, during which two gametes of opposite sex fuse to form a diploid zygote. The term generally includes reference to a pollen (including the sperm cell) and an ovule (including the ovum). “Crossing” therefore generally refers to the fertilization of ovules of one individual with pollen from another individual, whereas “selfing” typically defines the fertilization of ovules of an individual with pollen from the same individual. When referring to crossing in the context of achieving the introgression of a genomic region or segment, the skilled person will understand that in order to achieve the introgression of only a part of a chromosome of one plant into the chromosome of another plant, random portions of the genomes of both parental lines recombine during the cross due to the occurrence of crossing-over events in the production of the gametes in the parent lines. Therefore, the genomes of both parents must be combined in a single cell by a cross, where after the production of gametes from the cell and their fusion in fertilization will result in an introgression event.

The term “recipient”, as used herein, refers to the plant or plant line receiving the trait, transgenic event or genomic segment from a donor, and which recipient may or may not have the have trait, transgenic event or genomic segment itself either in a heterozygous or homozygous state.

The term “breeding line” or “elite line”, as used herein, refers to a line of a cultivated plant having commercially valuable or agronomically desirable characteristics, as opposed to wild varieties or varieties having beneficial qualities relating to experimental manipulation. The term includes reference to elite plant lines which represents an essentially homozygous, e.g. inbred or doubled haploid, line of plants used to produce F1 plants.

As used herein, the term “F1” means any offspring of a cross between two genetically unlike individuals.

The term “donor”, as used herein, refers to the plant or plant line from which the trait, transgenic event, or genomic segment originates, and which donor may have the trait, introgression or genomic segment in either a heterozygous or homozygous state.

The term “backcross”, as used herein, defines the crossing an F1 plant or plants with one of the original parents. A backcross is used to maintain the identity of one parent (species) and to incorporate a particular trait from a second parent (species). The term “backcross generation”, as used herein, refers to the offspring of a backcrossing.

The term “selfed”, as used herein, defines the crossing of two genetically identical plants. Typically, the term selfed defines self-pollination events and includes the fertilization process wherein both the ovule and pollen are from the same plant or plant line.

As used herein the term “recurrent parent percentage” relates to the percentage that a backcross progeny plant is identical to the recurrent parent plant used in the backcross. The percent identity to the recurrent parent can be determined experimentally by measuring genetic markers such as RFLPs or can be calculated theoretically based on a mathematical formula.

The term “offspring”, as used herein, refers to any progeny generation resulting from a crossing or selfing.

The term “identifying”, as used herein, refers to a process of establishing the identity or distinguishing character of a plant, such as exhibiting a certain trait.

The term “selecting”, as used herein, refers to a process of picking out a certain individual plant from a group of individuals, usually based on a certain identity of that individual.

The term “marker-assisted selection”, as used herein, refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “molecular marker”, as used herein, defines an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, Next Generation Sequencing (NGS) of a molecular marker, cleaved amplified polymorphic sequence (CAPS) markers or isozyme markers or combinations of the markers described herein which defines a specific genetic and chromosomal location.

The term “gene”, as used herein, refers to a hereditary unit consisting of a sequence of DNA that occupies a specific location on a chromosome and that contains the genetic instruction for a particular characteristics or trait in an organism. The term “gene” thus includes a nucleic acid (for example, DNA or RNA) sequence that comprises coding sequences necessary for the production of an RNA, or a polypeptide or its precursor. A functional polypeptide can be encoded by a full length coding sequence or by any portion of the coding sequence as long as the desired activity or functional properties (for example, enzymatic activity, ligand binding, signal transduction, etc.) of the polypeptide are retained.

### Embodiments

Backcross or pedigree selection is one method by which breeders add desirable agronomic traits to elite breeding lines. The method involves crossing the breeding line with a line that expresses the desirable trait followed by backcrossing offspring plants expressing the trait to the recurrent parent. The present disclosure is directed to improved methods of introgressing three or more desirable transgenic events into an elite plant germplasm. In one embodiment the method comprises preparing two donor lines in parallel wherein the donor lines comprise at least one shared desirable transgenic event to be transferred into the elite germplasm. The two donor lines are initially selected based on having certain desirable inheritable traits. The first and second donor lines are then crossed with an elite line to produce F1 progeny. The first and second F1 progeny are analyzed separately and plants from each of the two lines having the desired stacked transgenic events are selected and separately backcrossed with the recurrent parent to produce a first and second backcross progeny plants that have the desired trait and essentially all of the physiological and morphological characteristics of the original elite line. In one embodiment at least one of the backcross steps will be conducted using a female recurrent parent plant.

In one embodiment the final first and second donor plants produced by backcrossing with the recurrent parent will comprise the desired stacked transgenic events and will exhibit a high Recurrent Parent Percentage (RPP). The theoretical RPP can be calculated using a simple formula and based on the number of backcrosses conducted. If the parent used for backcrossing is homozygous, the recurrent parent percentage after N generations of backcrossing is 1−(½)^{N+1}×100. Six backcross generations are thus required to obtain greater than 99% recurrent parent percentage. Analysis of backcross progeny with Restriction Fragment Length Polymorphism (RFLP) markers is one method of analyzing the results and comparing the theoretical amount of inbreeding with actual levels of inbreeding observed. Additional molecular markers can also be used to assess RPP, including for example amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations thereof. Next Generation Sequencing (NGS) technology can also be used to cover the entire genome.

In one embodiment a method of introgressing three transgenic events into a recurrent plant comprises the preparation of two donor lines in parallel, wherein the two donor lines will be backcrossed to the same recurrent parent plant. The resulting two parallel backcrossed donor lines will differ in the desired transgenic events they contain, but will each comprise one transgenic event in common and both donors will having a Recurrent Parent Percentage of at least 75, 85, 88, 90, 94, 97 or 97.5%. In one embodiment the two parallel lines of donor plants will each comprise at least two transgenic events to be introgressed into the elite line and at least one of the transgenic events to be introgressed will be present in each of the two donor plants.

### Transgenic Event

The initial donor plant lines will comprise at least two desired transgenic events that are desired to be introgressed into an elite line. In an embodiment of the present disclosure the donor plants comprise two or more transgenic events wherein the transgenic events are not tightly linked and more typically are located on different chromosomes. In other embodiments, the donor plants comprise two or more transgenic events wherein the transgenic events are tightly linked on the same chromosome. Each transgenic event represents one or more desirable sequences that are sufficiently linked that they segregate together. In one embodiment the transgenic event is a locus comprising the genetic components that result in the expression of one or more traits. In one embodiment the transgenic event includes a series of regulatory sequences or genes, including for example, one or more inserted exogenous sequences that may contain one or more transgenes. The components comprising a transgenic event may comprise an open reading frame or a modified endogenous sequence. In one embodiment the transgenic event comprises one or more gene expression cassettes that further comprise actively transcribed and/or translated gene sequences. Conversely, the transgenic event may comprise a polynucleotide sequence which does not comprise a functional gene expression cassette or an entire gene (e.g., may simply comprise regulatory sequences such as a promoter), or may not contain any identifiable gene expression elements or any actively transcribed gene sequence.

In one embodiment the transgenic event comprises one or more genes encoding herbicide tolerance, insect resistance, nutrients, antibiotics or therapeutic molecules. In accordance with one embodiment the donor plant lines comprise multiple transgenic events “stacked” in the donor plant genome wherein the transgenic events comprise two or more genes encoding sequences that provide an agronomic trait. Examples of agronomic traits that can be stacked in the donor plant genome include, resistance or tolerance to glyphosate or another herbicide, and/or provides resistance to select insects or diseases and/or nutritional enhancements, and/or improved agronomic characteristics, and/or proteins or other products useful in feed, food, industrial, pharmaceutical or other uses. The “stacking” of two or more nucleic acid sequences (e.g., genes) of interest within a donor plant genome can be accomplished, for example, via conventional plant breeding using two or more events, transformation of a plant with a construct which contains the sequences of interest, re-transformation of a transgenic plant, or addition of new traits through targeted integration via homologous recombination.

Transgenic events in accordance with the present disclosure may include sequences including, but are not limited to, the following examples:

1. Genes or Coding Sequence (e.g. iRNA) that Confer Resistance to Pests or Disease

(A) Plant Disease Resistance Genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium fulvum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089).

(B) A Bacillus thuringiensis protein, a derivative thereof or a synthetic polypeptide modeled thereon, such as, a nucleotide sequence of a Bt δ-endotoxin gene (Geiser et al., 1986 Gene 48:109), and a vegetative insecticidal (VIP) gene (see, e.g., Estruch et al. (1996) Proc. Natl. Acad. Sci. 93:5389-94). Moreover, DNA molecules encoding δ-endotoxin genes can be purchased from American Type Culture Collection (Rockville, Md.), under ATCC accession numbers 40098, 67136, 31995 and 31998.

(C) A lectin, such as, nucleotide sequences of several Clivia miniata mannose-binding lectin genes (Van Damme et al., 1994 Plant Molec. Biol. 24:825).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al., 1987 J. Biol. Chem. 262:16793), a tobacco proteinase inhibitor I (Huub et al., 1993 Plant Molec. Biol. 21:985), and an α-amylase inhibitor (Sumitani et al., 1993 Biosci. Biotech. Biochem. 57:1243).

(F) An insect-specific hormone or pheromone such as an ecdysteroid and juvenile hormone a variant thereof, a mimetic based thereon, or an antagonist or agonist thereof, such as baculovirus expression of cloned juvenile hormone esterase, an inactivator of juvenile hormone (Hammock et al., 1990 Nature 344:458).

(G) An insect-specific peptide or neuropeptide which, upon expression, disrupts the physiology of the affected pest (J. Biol. Chem. 269:9). Examples of such genes include an insect diuretic hormone receptor (Regan, 1994), an allostatin identified in Diploptera punctata (Pratt, 1989), and insect-specific, paralytic neurotoxins (U.S. Pat. No. 5,266,361).

(H) An insect-specific venom produced in nature by a snake, a wasp, etc., such as a scorpion insectotoxic peptide (Pang, 1992 Gene 116:165).

(I) An enzyme responsible for a hyperaccumulation of monoterpene, a sesquiterpene, a steroid, hydroxamic acid, a phenylpropanoid derivative or another non-protein molecule with insecticidal activity.

(J) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al., 1993 Insect Molec. Biol. 23:691), and parsley ubi4-2 polyubiquitin gene (Kawalleck et al., 1993 Plant Molec. Biol. 21:673).

(K) A molecule that stimulates signal transduction. Examples of such molecules include nucleotide sequences for mung bean calmodulin cDNA clones (Botella et al., 1994 Plant Molec. Biol. 24:757) and a nucleotide sequence of a maize calmodulin cDNA clone (Griess et al., 1994 Plant Physiol. 104:1467).

(L) A hydrophobic moment peptide. See U.S. Pat. Nos. 5,659,026 and 5,607,914; the latter teaches synthetic antimicrobial peptides that confer disease resistance.

(M) A membrane permease, a channel former or a channel blocker, such as a cecropin-β lytic peptide analog (Jaynes et al., 1993 Plant Sci. 89:43) which renders transgenic tobacco plants resistant to Pseudomonas solanacearum.

(N) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al. (1990) Ann. Rev. Phytopathol. 28:451.

(O) An insect-specific antibody or an immunotoxin derived therefrom. Thus, an antibody targeted to a critical metabolic function in the insect gut would inactivate an affected enzyme, killing the insect. For example, Taylor et al. (1994) Abstract #497, Seventh Int'l. Symposium on Molecular Plant-Microbe Interactions shows enzymatic inactivation in transgenic tobacco via production of single-chain antibody fragments.

(P) A virus-specific antibody. See, for example, Tavladoraki et al. (1993) Nature 266:469, which shows that transgenic plants expressing recombinant antibody genes are protected from virus attack.

(Q) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al., 1992) Bio/Technology 10:1436. The cloning and characterization of a gene which encodes a bean endopolygalacturonase-inhibiting protein is described by Toubart et al. (1992 Plant J. 2:367).

(R) A developmental-arrestive protein produced in nature by a plant, such as the barley ribosome-inactivating gene that provides an increased resistance to fungal disease (Longemann et al., 1992). Bio/Technology 10:3305.

(S) RNA interference, in which an RNA molecule is used to inhibit expression of a target gene. An RNA molecule in one example is partially or fully double stranded, which triggers a silencing response, resulting in cleavage of dsRNA into small interfering RNAs, which are then incorporated into a targeting complex that destroys homologous mRNAs. See, e.g., Fire et al., U.S. Pat. No. 6,506,559; Graham et al. U.S. Pat. No. 6,573,099.

2. Genes that Confer Resistance to a Herbicide

(A) Genes encoding resistance or tolerance to a herbicide that inhibits the growing point or meristem, such as an imidazalinone, sulfonanilide or sulfonylurea herbicide. Exemplary genes in this category code for mutant acetolactate synthase (ALS) (Lee et al., 1988 EMBOJ. 7:1241) also known as acetohydroxyacid synthase (AHAS) enzyme (Miki et al., 1990 Theor. Appl. Genet. 80:449).

(B) One or more additional genes encoding resistance or tolerance to glyphosate imparted by mutant EPSP synthase and aroA genes, or through metabolic inactivation by genes such as DGT-28, 2mEPSPS, GAT (glyphosate acetyltransferase) or GOX (glyphosate oxidase) and other phosphono compounds such as glufosinate (pat, bar, and dsm-2 genes), and aryloxyphenoxypropionic acids and cyclohexanediones (ACCase inhibitor encoding genes). See, for example, U.S. Pat. No. 4,940,835, which discloses the nucleotide sequence of a form of EPSP which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061. European patent application No. 0 333 033 and U.S. Pat. No. 4,975,374 disclose nucleotide sequences of glutamine synthetase genes which confer resistance to herbicides such as L-phosphinothricin. The nucleotide sequence of a phosphinothricinacetyl-transferase gene is provided in European application No. 0 242 246. De Greef et al. (1989) Bio/Technology 7:61 describes the production of transgenic plants that express chimeric bar genes coding for phosphinothricin acetyl transferase activity. Exemplary of genes conferring resistance to aryloxyphenoxypropionic acids and cyclohexanediones, such as sethoxydim and haloxyfop, are the Accl-S1, Accl-S2 and Accl-S3 genes described by Marshall et al. (1992) Theor. Appl. Genet. 83:435.

(C) Genes encoding resistance or tolerance to a herbicide that inhibits photosynthesis, such as a triazine (psbA and gs+ genes) and a benzonitrile (nitrilase gene). Przibilla et al. (1991) Plant Cell 3:169 describe the use of plasmids encoding mutant psbA genes to transform Chlamydomonas. Nucleotide sequences for nitrilase genes are disclosed in U.S. Pat. No. 4,810,648, and DNA molecules containing these genes are available under ATCC accession numbers 53435, 67441 and 67442. Cloning and expression of DNA coding for a glutathione S-transferase is described by Hayes et al. (1992) Biochem. J. 285:173.

(D) Genes encoding resistance or tolerance to a herbicide that bind to hydroxyphenylpyruvate dioxygenases (HPPD), enzymes which catalyze the reaction in which para-hydroxyphenylpyruvate (HPP) is transformed into homogentisate. This includes herbicides such as isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular 2-cyano-3-cyclopropyl-1-(2-SO2CH3-4-CF3 phenyl)propane-1,3-dione and 2-cyano-3-cyclopropyl-1-(2-SO2CH3-4-2,3Cl2phenyl)propane-1,3-dione, triketones (EP625505, EP625508, U.S. Pat. No. 5,506,195), in particular sulcotrione, and pyrazolinates. A gene that produces an overabundance of HPPD in plants can provide tolerance or resistance to such herbicides, including, for example, genes described in U.S. Pat. Nos. 6,268,549 and 6,245,968 and U.S. Patent Application, Publication No. 20030066102.

(E) Genes encoding resistance or tolerance to phenoxy auxin herbicides, such as 2,4-dichlorophenoxyacetic acid (2,4-D) and which may also confer resistance or tolerance to aryloxyphenoxypropionate (AOPP) herbicides. Examples of such genes include the α-ketoglutarate-dependent dioxygenase enzyme (aad-1) gene, described in U.S. Pat. No. 7,838,733.

(F) Genes encoding resistance or tolerance to phenoxy auxin herbicides, such as 2,4-dichlorophenoxyacetic acid (2,4-D) and which may also confer resistance or tolerance to pyridyloxy auxin herbicides, such as fluroxypyr or triclopyr. Examples of such genes include the α-ketoglutarate-dependent dioxygenase enzyme gene (aad-12), described in WO 2007/053482 A2.

(G) Genes encoding resistance or tolerance to dicamba (see, e.g., U.S. Patent Publication No. 20030135879).

(H) Genes providing resistance or tolerance to herbicides that inhibit protoporphyrinogen oxidase (PPO) (see U.S. Pat. No. 5,767,373).

(I) Genes providing resistance or tolerance to triazine herbicides (such as atrazine) and urea derivatives (such as diuron) herbicides which bind to core proteins of photosystem II reaction centers (PS II) (See Brussian et al., (1989) EMBO J. 1989, 8(4): 1237-1245.

3. Genes that Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al., 1992) Proc. Nat. Acad. Sci. USA 89:2624.

(B) Decreased phytate content

(1) Introduction of a phytase-encoding gene, such as the Aspergillus niger phytase gene (Van Hartingsveldt et al., 1993 Gene 127:87), enhances breakdown of phytate, adding more free phosphate to the transformed plant.

(2) A gene could be introduced that reduces phytate content. In maize, this, for example, could be accomplished by cloning and then reintroducing DNA associated with the single allele which is responsible for maize mutants characterized by low levels of phytic acid (Raboy et al., 1990 Maydica 35:383).

(C) Modified carbohydrate composition effected, for example, by transforming plants with a gene coding for an enzyme that alters the branching pattern of starch. Examples of such enzymes include, Streptococcus mucus fructosyltransferase gene (Shiroza et al., 1988) J. Bacteriol. 170:810, Bacillus subtilis levansucrase gene (Steinmetz et al., 1985 Mol. Gen. Genel. 200:220), Bacillus licheniformis α-amylase (Pen et al., 1992 Bio/Technology 10:292), tomato invertase genes (Elliot et al., 1993), barley amylase gene (Sogaard et al., 1993 J. Biol. Chem. 268:22480), and maize endosperm starch branching enzyme II (Fisher et al., 1993 Plant Physiol. 102:10450).

In one embodiment the transgenic event comprises one or more transgenes selected from the group consisting of an insecticidal resistance transgene, herbicide tolerance transgene, nitrogen use efficiency transgene, water use efficiency transgene, nutritional quality transgene, DNA binding transgene, and selectable marker transgene.

### Donor Plants

In accordance with one embodiment donor plants are selected that comprise a stacked set of at least two transgenic events. These donor plants can be generated using standard recombinant or breeding techniques or any combination thereof. In one embodiment the donor plant comprises 2, 3, 4, 5, 6, or more stacked transgenic events. Furthermore, each transgenic event may comprise multiple components that are tightly linked and segregate together. In one embodiment each transgenic event comprises a series of genes that are related in function or are associated with a particular desired trait. The donor lines have genetic backgrounds that assist in the creation of plants having the transgenes. However, to fully capture the benefit of the traits associated with the transgenic events, the transgenic events must be introgressed into elite breeding lines.

In accordance with one embodiment a method is provided to reduce the number of crosses required in a traditional backcross selection methodology. The method comprises preparing two donor plant lines in parallel wherein the transgenic events desired for introgression are distributed between the two donor plants with at least one transgene being in common between the two donor plants. In one embodiment a first donor plant comprises a first and second transgenic event and the second donor plant comprises the same second transgenic event and a third transgenic event. In one embodiment a first donor plant comprises a first, second and third transgenic event and the second donor plant comprises the same third transgenic event and a fourth transgenic event. In one embodiment a first donor plant comprises a first, second and third transgenic event and the second donor plant comprises the same third transgenic event and a fourth and fifth transgenic event. In one embodiment a first donor plant comprises a first, second, third and fourth transgenic event and the second donor plant comprises the same third and fourth transgenic event and a fifth and sixth transgenic event.

In accordance with one embodiment the first and second donor plants are maintained as two separate lines, with each line being backcrossed with the same recurrent parent line to generate a first and second donor lines each having a high recurrent parent percentage to the same recurrent line. More particularly, in one embodiment the first and second donor plants are each crossed with same elite plant line and the first and second F1 progeny of the two crosses that retain the desired transgenic events are further backcrossed with the recurrent parent line to generate first and second backcross progeny plants that comprise the desired traits and essentially all of the physiological and morphological characteristics of the elite line, except for the characteristics derived from the desired transgenic events. The resulting first donor/recurrent plant can then be crossed with said second donor/recurrent plant to produce progeny having the desired transgenic events fixed in the desired elite plant germplasm.

In accordance with one embodiment a method for introgressing three or more transgenic events from donor lines into a single elite plant germplasm is provided. The method comprises

a) providing a first donor/recurrent plant comprising a first transgenic event, a second transgenic event and having a recurrent parent percentage greater than 80%, 88%, 90%, 94%, 97% or 97.5%;

b) providing a second donor/recurrent plant comprising said second transgenic event, a third transgenic event and having a recurrent parent percentage greater than 80%, 88%, 90%, 94%, 97% or 97.5%;

c) crossing said first donor/recurrent plant with said second donor/recurrent plant to produce progeny;

d) identifying and selecting product plants from the plants grown in step (c) or optionally selfed offspring of the plants of step (c), that comprise said first, second and third transgenic events.

In accordance with one embodiment the first and second donor/recurrent plant are generated by crossing an initial first and second donor plant with an elite line to generate first and second lines of F1 progeny. One or more progeny plants from the first and second F1 progeny are then selected based on having the desired trait and further crossed in parallel (i.e., the two first and second F1 progeny are propageted as two separate lines) with the recurrent parent plants to produce backcross progeny plants; the backcross progeny plants are then selected for those that have the desired trait and the backcrossing steps are repeated one, two or more times to produce selected second, third or higher backcross progeny plants that comprise the desired traits and essentially all of the physiological and morphological characteristics of the elite line, except for the characteristics derived from the donor plant. In accordance with one embodiment the recurrent parent plant used in the backcrossings is a female plant for at least one of the backcrosses.

In accordance with one embodiment progeny plants are screened and selected for those comprising the desired traits through the use of marker assisted selection. In one embodiment the marker assisted selection techniques used is selected from the group consisting of SNP marker assisted selection, SSR marker assisted selection, RFLP marker assisted selection, RAPD marker assisted selection, and AFLP marker assisted selection. Next Generation Sequencing (NGS) technology can also be used to cover the entire genome during backcrossing.

In accordance with one embodiment the backcross donor plants comprise genomic sequences that share over 80%, 88%, 90%, 94%, 97%, or 97.5% sequence identify with the recurrent parent line. In accordance with one embodiment the first and second backcross donor plants comprise less than 5 cM, 10 cM, 20 cM, 25 cM, 30 cM, 35 cM, 40 cM, 45 cM, or 50 cM linkage drag from the first or second donor parent plant, respectively. In accordance with one embodiment the backcross donor plants comprise the desired transgenic events with greater than 88%, 90%, 95%, 97%, or 97.5% molecular markers in common with the recurrent parent plant. In one embodiment the backcross donor plants comprise the desired transgenic events with essentially all of the physiological and morphological characteristics of the elite line.

In accordance with one embodiment a first donor plant, comprising a first stack of at least two transgenic events is provided, and that first donor plant is crossed with a selected recurrent parent plant to produce first F1 progeny plant that comprises said first stack of transgenic events. A first breeding backcross is made of the first F1 progeny plants with the recurrent parent plant, and a first breeding backcross progeny plant is selected comprising said first stack of transgenic events. The first breeding backcross progeny are then backcrossed with the recurrent parent plant one or more times in succession to produce a BC2, BC3 or BC4 or higher first backcross progeny plant comprising the first stack of transgenic events. In accordance with one embodiment one or more of the backcrosses is conducted using a female recurrent plant.

In a similar fashion a second donor plant is selected comprising a second stack of at least two transgenic events, wherein at least one of the transgenic events of said second stack is also present in the first stack of transgenic events in the first donor plant. The second donor plant donor plant is crossed with the same selected recurrent parent plant used to produce the first F1 progeny plants. This cross produces second F1 progeny plants that comprises said second stack of transgenic events. A second breeding backcross is made of the second F1 progeny plants with the recurrent parent plant, and a second breeding backcross progeny plant is selected comprising said second stack of transgenic events. The second breeding backcross progeny are then backcrossed with the recurrent parent plant one or more times in succession to produce a BC2, BC3 or BC4 or higher second backcross progeny plant comprising the second stack of transgenic events. In accordance with one embodiment one or more of the backcrosses is conducted using a female recurrent plant.

The BC2, BC3 or BC4 or higher first and second backcross progeny plants will have a high recurrent parent percent with greater than 88%, 90%, 95%, 97% or 97.5% molecular markers in common with the recurrent parent plant and/or less than 20 cM linkage drag from the first or second donor parent plant, respectively. The BC2, BC3 or BC4 or higher first backcross progeny are then crossed with the BC2, BC3 or BC4 or higher second backcross progeny to produce a third progeny plant comprising the unique three (or more) transgenic events from the first and second stacks of transgenic events. Optionally the third progeny plant is selfed or backcrossed with the recurrent parent while selecting for those progeny plants that comprise the desired introgressed transgenic events. In one embodiment the third progeny plant has a recurrent parent percent of at least 97.5%. In one embodiment one or more of the transgenic events is fixed in the final breeding line in a homozygous state. In one embodiment all of the transgenic events are fixed in the homozygous state. In one embodiment the plants comprising the introgressed desired transgenic events and recurrent parent germplasm comprises less than 5 cM, 10 cM, 15 cM, 20 cM, 25 cM, 30 cM, 35 cM, 40 cM, 45 cM, or 50 cM linkage drag from the first and/or second donor parent plant.

In accordance with one embodiment plants comprising the desired transgenic events and recurrent parent germplasm can be crossed with a closely related species rather than the same species. For example in one embodiment, maize plants can be crossed with related plants such as teosinte. Alternatively, or additionally, in one embodiment the first and second donor plants comprising the desired transgenic events can be from closely related but not identical species.

In accordance with one embodiment the present method of introgressing three or more transgenic events into an elite line can be used on any plant species that can be bred by backcross selection. In one embodiment the plant is a crop species and can be selected from monocots or dicots. For example, monocot plants for use in accordance with the present disclosure comprise any plant selected from the group consisting of a corn plant, a wheat plant, or a rice plant. Furthermore specific examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats (Avena), barley (Hordeum), pineapple (Ananas comosus), banana (Musa spp.), palm, ornamentals, and grasses. In one embodiment the moncot is corn. Examples of dicot plants for use in accordance with the present disclosure comprise any plant selected from the group consisting of a soybean plant, a tomato plant, an alfalfa plant, a canola plant, a rapeseed plant, a Brassica plant, a cotton plant, and a sunflower plant. Additional examples of dicot plants that can be used in accordance with the present disclosure include, but are not limited to, canola, cotton, potato, quinoa, amaranth, buckwheat, safflower, soybean, sugarbeet, sunflower, canola, rapeseed, tobacco, Arabidopsis, Brassica, and cotton. In one embodiment the dicot is soybean.

### Example 1

### Marker Assisted Trait Conversion of RRH63 Inbred with Mon88017:Mon89034 in Corn SmartStax 8

SmartStax 8 (SSX 8) is a corn plant generated by introgressing 8 genes [TC1507 (PAT, Cry 1F), DAS591227 (PAT, Cry34Ab1, Cry35Ab1), Mon89034 (Cry 1A.105, Cry2Ab2), Mon88017 (EPSPS, Cry3Bb1)] into high yielding corn lines through marker assisted backcross breeding. The ultimate goal of SmartStax 8 is to protect corn from major pests such as cornborers, rootworms, cutworms, earworms and armyworms. SmartStax 8 will also provide resistance to glyphosate herbicide and incorporate glufosinate as a selectable marker.

Marker Assisted Backcross Breeding (MABB)

Crop improvement through classical breeding methods has made remarkable progress during the past century. However, considering the steady increase of the human population and the ever shrinking land available for crop cultivation, it is imperative to adopt new strategies to meet the demands. Development of high yielding varieties with desirable traits in a short time span is an important focus of breeding programs. Marker assisted selection (MAS) will facilitate development of superior cultivars in a shorter time than the classical approaches. Availability of genomic resources such as neutral (DNA based) molecular markers (especially co-dominant markers) is necessary in order to take advantage of MAS. Backcross breeding has been a common method to incorporate one or a few genes from a donor into an adapted variety that has been used in plant breeding for nearly a century. Although trait introgression can be achieved through traditional backcross breeding methods, it takes up to 6 generations to obtain 99% recurrent parent percentage (RPP). However, MAS expedites the recovery of more than 99% RPP in just three backcross generations which indicates that molecular markers increase the efficiency of backcrossing tremendously (Bassett et al. 2000).

Goal

Introgression of Mon89034 and Mon88017 genes into corn elite inbred SMA07BM through marker assisted backcross breeding which eventually will be stacked with Mon88017 and TC1507 genes at BC3 generation to obtain SMA07BM with all three genes Mon88017::Mon89034::TC1507 and recover more than 98% recurrent parent genome (RPP)

Conversion Strategy

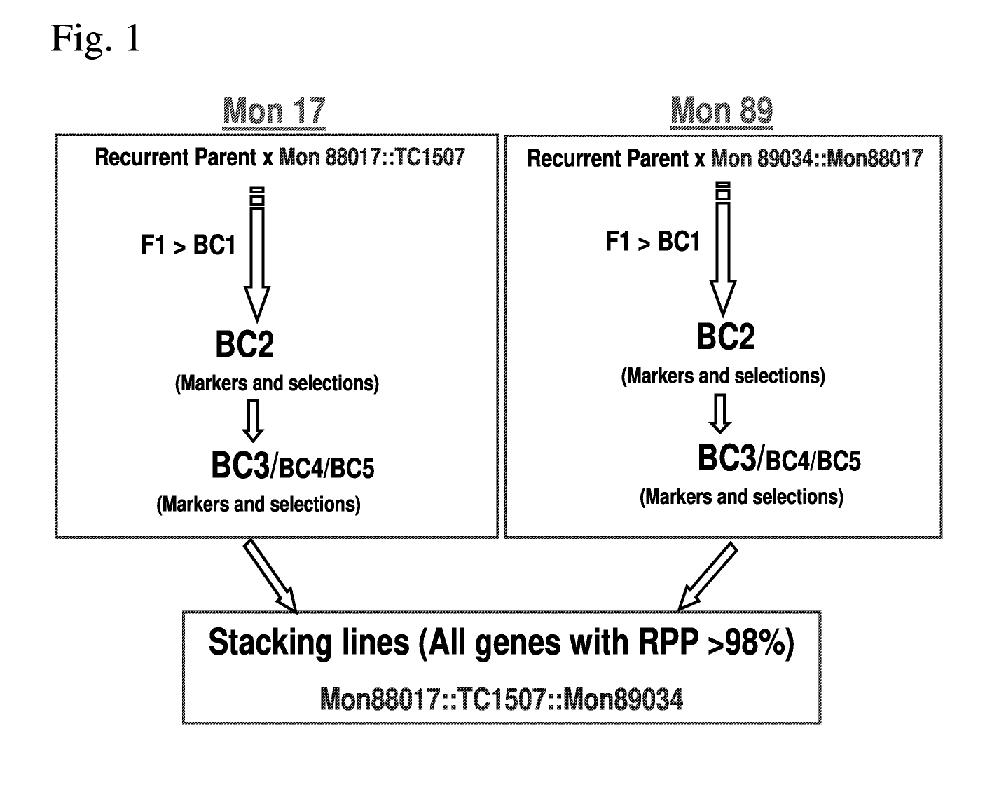

In the female side of SSX8, the focus was to stack Mon88017 and Mon89034 along with TC1507 into an elite germplasm and recover 98% or more recurrent parent genome. In order to do that, parallel conversions for Mon17 (Mon88017::TC1507) and Mon89 (Mon88017::Mon89034) were run separately and a strategy of marker assisted selection starting from BC2 and BC3 and stacking at BC3 (see FIG. 1). In cases when the stacking at BC3 is not achievable because of unforeseen reasons, the lines will be stacked at BC4 or BC5. Alternatively, this strategy could also be run by using markers on the BC1 and BC2 and stacking at the BC2. Since the Mon88017 is present in both sides, we were able to select stacked lines that had an allelic status of Homo::Hemi::Hemi for Mon88017::TC1507::Mon89034, respectively.

### Materials and Methods

### BC2

DNA Extraction and Preparation

422 samples from the BC2 RRH63 Mon88017::Mon89034 population were sampled. DNA was extracted from fresh tissue using the MagAttract™ protocol on the Agilent Biocel Robot™ (Bohl et al 2010). The samples were tested by High Through-Put Molecular Analysis (HTMA) for the presence of Mon89034 (Hinchey 2002). Next, 188 DNA samples testing Hemi for Mon89034 were hit-picked into 2, 96-well plates.

The DNA was diluted at a 1:10 ratio with distilled water. Next, 6RC162 Mon88017::Mon89034, the population donor and RRH63, the Recurrent Parent, were taken from MABL stock DNA and added to wells A1 and A2 in Plate 1 and wells H10 and H11 in Plate 2.

SNP Markers and Genotyping Platform

Marker analysis was performed using KASPar™ SNP platform. The marker selections are listed below in Table 1. The resulting data was uploaded and analyzed using Kraken Kluster Caller™ software. Marker Study Manager was used to assemble a chromosome table and calculate Recurrent Parent Percentage (RPP) for each sample.

Linkage Drag Analysis

To carry out Linkage Drag (LD) analysis, polymorphic markers that amplified loci across the entire genome were selected using markers. LD was performed on chromosome 1 and 4, because event Mon89034 was previously mapped to chromosome 1 around 342 cM, and event Mon88017 was previously mapped to chromosome 4 around 110 cM. The selected markers were spaced approximately 10 cM apart on chromosome 1 and 4.

Genome Analysis

The 45 samples with the highest RPP were hit-picked from the extraction plate into one 96-well plate for the HTMA lab to check for presence of Mon88017, and into another to run Genome Analysis (GA). The GA was performed on chromosomes 2-3, 5-10 using KASPar™ SNP analysis. The selected markers were spaced approximately 20 cM apart, and are listed below in Table 1. The resulting data was uploaded and analyzed using Kraken Kluster Caller™ software. Marker Study Manager™ was used to assemble a chromosome table that includes both the previous LD and the new GA analysis and to calculate Recurrent Parent Percentage (RPP) for each sample.

The 10 plants with the highest RPP and testing as hemizygous for both Mon88017 and Mon89034 were selected.

### BC3

DNA Extraction and Preparation

Next, 188 samples from the BC3 RRH63 Mon88017::Mon89034 population were sampled along with RRH63, the Recurrent Parent. Then DNA was extracted from fresh tissue using the MagAttract™ protocol on the Agilent Biocel Robot™. The extracted DNA was diluted at a 1:10 ratio with distilled water. The 6RC172 Mon88017::Mon89034, the population donor, was taken from stock DNA and added to well A2 in Plate 1 and to H11 in Plate 2.

SNP Markers and Genotyping Platform

The BC3 marker analysis was performed using KASPar™ SNP analysis. The marker selections are listed below in Table 1. The resulting data was uploaded and analyzed using Kraken Kluster Caller™ software. Marker Study Manager™ was used to assemble a chromosome table and to calculate Recurrent Parent Percentage (RPP) for each sample.

Linkage Drag and Genome Analysis

Polymorphic markers that amplified loci across the entire genome were selected using MarkerDB™. The LD was performed on chromosome 1 and 4 because event Mon89034 was previously mapped to chromosome 1 around 342 cM, and event Mon88017 was previously mapped to chromosome 4 around 110 cM. The selected markers were spaced approximately 10 cM apart on chromosome 1 and 4 and approximately every 20 cM throughout the rest of the genome. The genotype of plants from BC2 used as donors for the BC3 generation, were assessed. Markers homozygous for the A allele in all BC3 population donors were eliminated from the analysis and added to final results as historical, non-segregating markers.

The 10 plants with the top RPP were selected. The top selections for both the Mon88017::Mon89034 population and the Mon88017::TC1507 populations were entered into a stacking program to find the best possible stacking combinations.

TABLE 1
Markers, along with locus chromosome and Chromosome Position, used for
KASPar ™ SNP platform in BC2 linkage drag analysis, BC2 genome analysis, and BC3
linkage drag/genome analysis.

| BC2 LD | | | BC2 GA | | | BC3 LDGA | | |
|---|---|---|---|---|---|---|---|---|
| Marker | | chr_position | Marker | | chr_position | marker | | chr_position |
| (snp_id) | chr | cM | (snp_id) | chr | cM | (snp_id) | chr | cM |
| 5177 | 1 | 168.74 | 14651 | 1 | 243.15 | 9017 | 1 | 75.68 |
| 2077 | 1 | 174.98 | 7500 | 4 | 72.08 | 4295 | 1 | 142.1 |
| 9678 | 1 | 206.86 | 6900 | 4 | 88.41 | 7915 | 1 | 218.89 |
| 5535 | 1 | 211.75 | 6849 | 4 | 88.62 | 5177 | 1 | 230.72 |
| 5438 | 1 | 215.08 | 1583 | 5 | 105.67 | 9678 | 1 | 283.6 |
| 5253 | 1 | 225.34 | 12832 | 5 | 125.31 | 9232 | 1 | 295.12 |
| 12658 | 1 | 228.32 | 9746 | 5 | 148.99 | 8905 | 1 | 309.83 |
| 14651 | 1 | 243.15 | 4882 | 6 | 58.68 | 14141 | 1 | 319.44 |
| 3266 | 2 | 78.33 | 9272 | 6 | 76.08 | 3524 | 1 | 360.98 |
| 8930 | 2 | 138.04 | 1628 | 6 | 97.75 | 1492 | 2 | 192.8 |
| 2542 | 2 | 157.56 | 13922 | 6 | 108.16 | 2542 | 2 | 210.64 |
| 14157 | 4 | 0 | 12143 | 6 | 124.36 | 8872 | 2 | 259.35 |
| 7375 | 4 | 7.76 | 5479 | 7 | 68.96 | 5221 | 3 | 89.25 |
| 2479 | 4 | 16.33 | 12961 | 7 | 84.86 | 1204 | 3 | 110.87 |
| 9466 | 4 | 33.53 | 14175 | 7 | 100.15 | 12125 | 3 | 118.19 |
| 13701 | 4 | 40.96 | 13058 | 7 | 120.08 | 11448 | 3 | 253.43 |
| 10513 | 4 | 70.89 | 8781 | 7 | 139.36 | 5782 | 3 | 263.5 |
| 6900 | 4 | 88.41 | 4958 | 7 | 148 | 4784 | 4 | 5.29 |
| 9332 | 4 | 90.12 | 14598 | 8 | 44.96 | 13701 | 4 | 46.35 |
| 14281 | 4 | 122.62 | 5615 | 8 | 62.61 | 10513 | 4 | 98.21 |
| 11532 | 4 | 122.92 | 3663 | 8 | 75.17 | 7500 | 4 | 101.23 |
| 5567 | 4 | 170.75 | 8832 | 8 | 93.97 | 3891 | 4 | 148.7 |
| | | | 7215 | 8 | 109.14 | 1334 | 4 | 153.5 |
| | | | 2676 | 9 | 1.13 | 5567 | 4 | 212.99 |
| | | | 12620 | 9 | 11.54 | 12067 | 5 | 137.77 |
| | | | 6964 | 9 | 50.5 | 1583 | 5 | 151.53 |
| | | | 7594 | 9 | 68.41 | 11143 | 5 | 181.55 |
| | | | 9026 | 9 | 152.24 | 11187 | 6 | 87.16 |
| | | | 3377 | 10 | 68.72 | 4290 | 6 | 95.1 |
| | | | 4448 | 10 | 82.34 | 9272 | 6 | 110.39 |
| | | | | | | 8473 | 6 | 112.73 |
| | | | | | | 6585 | 6 | 132.07 |
| | | | | | | 1628 | 6 | 134.07 |
| | | | | | | 2784 | 6 | 143.85 |
| | | | | | | 1433 | 7 | 98.72 |
| | | | | | | 5162 | 7 | 110.05 |
| | | | | | | 6876 | 7 | 131.44 |
| | | | | | | 3036 | 7 | 148 |
| | | | | | | 1171 | 7 | 161.06 |
| | | | | | | 2356 | 7 | 167.25 |
| | | | | | | 5545 | 7 | 191.69 |
| | | | | | | 9512 | 8 | 60.38 |
| | | | | | | 5615 | 8 | 85.76 |
| | | | | | | 3663 | 8 | 103.3 |
| | | | | | | 12689 | 8 | 119.5 |
| | | | | | | 13259 | 8 | 129.11 |
| | | | | | | 11806 | 9 | 9.6 |
| | | | | | | 4132 | 9 | 38.29 |
| | | | | | | 8959 | 9 | 47.92 |
| | | | | | | 1971 | 9 | 87.06 |
| | | | | | | 2537 | 10 | 23.14 |

Stacking

A parallel backcross conversion of RRH63 with Mon 17::TC1507 was performed as shown in FIG. 1. The goal was to stack both sides of RRH63 and to obtain a stacked combination which will result in a line that will have greater than 98% RPP. A stacking combination program was run using the top 10 selections of Mon 17 and Mon 89 sides to determine statistically the probability of obtaining a combination that will result in getting >98% RPP for all combinations. The marker analysis of the resulting stacked population was completed.

Phenotyping

New Start Nurseries

This nursery is the initial step in transferring the trait of interest into the target recurrent parent plant (RPP) inbred line. All potential donor plants were screened for gene expression using a quantitative ELISA test. Only high expressing donor plants were used to pollinate into the recurrent parent. Pollen should be carried from the donor to the RPP in order to capture RPP cytoplasm. A minimum of 10 pollinations were made and those ears were bulk-shelled.

F1 to BC1 Nursery

Every F1 population was tested to confirm that the gene(s) of interest were present. This was done by spraying the appropriate selectable herbicide marker (Round-Up® for Mon 88017 or Liberty Link® for TC1507) or using a qualitative ELISA test (Mon 89034). If no errors were made, all plants tested should have the gene(s) of interest. At flowering, a minimum of 10 pollinations were made by carrying pollen from the RPP into the F1 material.

BC1 and BC2 Nursery

Here again, and in all subsequent nurseries, selectable herbicide markers and qualitative ELISA were performed. Plants will segregate for the presence of each gene in a 1:1 ratio. At flowering, 20 positive plants were selected and pollinated. At harvest, 10 of those plants were selected based on ear type and were bulked shelled.

BC2 to BC3 Nursery

This was the first generation where the population were genotyped. It was necessary to plant enough rows to be able to obtain at least 186 positive plants after gene presence (through herbicide application or qualitative ELISA) was assayed. Leaf tissue samples were collected on 3-4 week olds plants. The lab chose the top plants by flowering and only those plants were used in pollinations. At this stage, the top plants were used in two ways: they were pollinated by the RPP to make a reverse backcross and they were used to pollinate into the RPP to make up to 5 backcross pollinations. The pollinations made using the top 10 plants from every population were harvested, but only the best 3 plants were replanted. Phenotypic selection was used to complement the marker selections in the BC2, discarding only those plants with major phenotypic problems.

BC3 Stacking Nurseries

With the growing number of genes to be stacked, stacking designs are becoming increasingly complex. When 3 or more traits are to be introgressed into a line, the introgression optimally should to be separated into two different marker introgression projects initially and the final gene combination is achieved through stacking. Typically, stacking is performed at the BC3 (or later, e.g., BC4) generation, using plants that are hemizygous for their respective genes.

When stacking is performed using BC3 (hemizygous) plants, it is important to receive a full genome analysis of the plants to be used in stacking before pollination. The specific crossing combinations are to be determined through a meeting with the lab group after the genome analysis results are available. Lines to be stacked must be selected so that they have “complementary” donor parent background introgression. This will allow the near-complete elimination of the donor genetic background with the help of markers on S1 plants later on.

### Results

Genotyping Data to Assist Quantitative Genetics in Making Selections LD BC2

In a few loci, the RPP (sent from field) and donor alleles were monomorphic, yet the population was segregating. The allele call for the RPP was forced to heterozygous in KLIMs for these instances—to match the expected call and to match the population segregation. In most cases in which the parents were polymorphic, there were BBs present in the population. The genotypes in the field most likely result from a dirty RPP used at an earlier generation. Low marker coverage and no right flanking marker for Mon89 on Chr 1. Data return rate was 99.1%. The top 45 plants were selected for pollination and zygosity testing.

GA BC2

Data return rate was 98.2%. Several markers were dropped due to strange population segregation—particularly around cM 110 on Chr4 and on Chr9.

LDGA BC3

The data rate was 99.1%. The following plants; 94305117, 94308841, 94308798 were rogue samples. No markers to the right of Mon 89. Unexpected segregation was observed in the rogue plants; all other family groups match historical segregation patterns.

Segregating Markers 7594, 2479 did not appear as polymorphic markers for this cross (a different source of RPP was provided between BC2 and BC3). The top 10 plants were selected by flowering for stacking. Also, if more than 10 plants had 100% RPP, they were selected so that the plants could be chosen phenotypically.

Selections

The top plant selections along with their RPP's are listed below in Table 2 for BC2 and BC3 LDGA. The top stacking combinations are listed in Table 3.

TABLE 2
Final 10 plant picks from RRH63 Mon88017::Mon89034
populations communicated to breeders for BC2 and
BC3 generations, along with the Recurrent Parent
Percentage and Selection Rank for each plant.

| BC2 | | | BC3 | | |
|---|---|---|---|---|---|
| | | % | | | % |
| | % | Recurrent | | % | Recurrent |
| Sample Tube | Recurrent | Parent | | Recurrent | Parent |
| Number | Parent | Rank | Row Id | Parent | Rank |
| 89955732 | 91.6 | 1 | 8320360 | 2 | 90.8 |
| 89956229 | 91.5 | 3 | 8320402 | 3 | 95.7 |
| 89956397 | 91.4 | 1 | 8320408 | 1 | 96.6 |
| 89955623 | 87.7 | 1 | 8320462 | 1 | 94.4 |
| 89955658 | 87 | 2 | 8320498 | 3 | 98.2 |
| 89955719 | 86.5 | 3 | 8320512 | 1 | 96.7 |
| 89955561 | 86.3 | 3 | 8320550 | 3 | 98 |
| 89137769 | 83.8 | 2 | 8320637 | 2 | 95.9 |
| 89138637 | 83.1 | 3 | 8320679 | 3 | 88.1 |
| 89955672 | 83 | 3 | 8320687 | 3 | 90.9 |

Stacking Combination

A stacking combination program using the top 10 selections of Mon 17 and Mon 89 sides determined statistically the probability of obtaining a combination that will result in getting >98% RPP for all combinations. The highest probable combinations were used in the field for making the actual cross. Table 3 indicates the stacking combinations that were used in the field.

TABLE 3
The top stacking combinations, shown as Plant_Numbers, paired together
from each of the conversions (Mon88017:TC1507 and Mon89034: Mon88017)

| Mon89034: Mon88017 | Mon88017:TC1507 | |
|---|---|---|
| conversion | conversion | Mon88017:TC1507 conversion |
| plant number | plant number | Pedigree |
| 682 | 5391 | RRH63[2]/Mon 88017::TC1507=B=B |
| 734 | 5424 | RRH63[2]/Mon 88017::TC1507=B=B |
| 734 | 5524 | RRH63[2]/Mon 88017::TC1507=B=B |
| 513 | 5401 | RRH63[2]/Mon 88017::TC1507=B=B |
| 696 | 5469 | RRH63[2]/Mon 88017::TC1507=B=B |
| 682 | 5496 | RRH63[2]/Mon 88017::TC1507=B=B |
| 734 | 5400 | RRH63[2]/Mon 88017::TC1507=B=B |
| 696 | 5466 | RRH63[2]/Mon 88017::TC1507=B=B |
| 682 | 5445 | RRH63[2]/Mon 88017::TC1507=B=B |
| 734 | 5441 | RRH63[2]/Mon 88017::TC1507=B=B |

| Mon88017:TC1507 | Mon89034: Mon88017 | |
|---|---|---|
| conversion | conversion | Mon89034: Mon88017 conversion |
| Plant_Number | Plant_Number | Pedigree |
| 5391 | 696 | RRH63[3]/Mon 88017::Mon 89034=B=B=B.1796 |
| 5401 | 513 | RRH63[3]/Mon 88017::Mon 89034=B=B=B.1714 |
| 5524 | 646 | RRH63[3]/Mon 88017::Mon 89034=B=B=B.1796 |
| 5524 | 465 | RRH63[3]/Mon 88017::Mon 89034=B=B=B.1714 |
| 5424 | 507 | RRH63[3]/Mon 88017::Mon 89034=B=B=B.1714 |
| 5424 | 883 | RRH63[3]/Mon 88017::Mon 89034=B=B=B.1990 |
| 5391 | 933 | RRH63[3]/Mon 88017::Mon 89034=B=B=B.1990 |
| 5401 | 925 | RRH63[3]/Mon 88017::Mon 89034=B=B=B.1990 |
| 5391 | 682 | RRH63[3]/Mon 88017::Mon 89034=B=B=B.1796 |
| 5401 | 734 | RRH63[3]/Mon 88017::Mon 89034=B=B=B.1796 |

### Discussion

The RRH63×SLB01 conversion was a narrow cross, and as a result relatively few polymorphic markers were available for use in analysis. It is assumed that most areas of the genome without polymorphic marker coverage do not differ between the two lines and do not necessitate conversion. The RPP progress from BC2 to BC3 was significant and within the 98% minimum RPP set for stacking.

Selfing and Testcrossing Nursery

This was the last generation to be planted. This nursery was used to advance project material from the S1 to the S2 level of selfing while concurrently producing hybrid seed for yield trial testing in the target environment. Typically, all seeds obtained from the top 5 S1 plants were planted and these plants formed 5 selections to be used in yield trials.

All plants in this nursery were assayed for zygosity. At pollination, plants that were homozygous for both Mon88107 and Mon 89034 and either homozygous or null for TC1507 were self-pollinated to send to the finishing nursery. Other plants in the population were used as female to produce hybrid seed by carrying pollen in from a male tester.

1. A method for introgressing three or more transgenic events from donor lines into a recurrent plant genome, said method comprising:
a) providing a first donor/recurrent plant comprising a first transgenic event, a second transgenic event and having a recurrent parent percentage of greater than 80%;
b) providing a second donor/recurrent plant comprising said second transgenic event, a third transgenic event and having a recurrent parent percentage of greater than 80%;
c) crossing said first donor/recurrent plant with said second donor/recurrent plant to produce progeny plants;
d) identifying and selecting progeny plants from those in step (c), or optionally selfed offspring of the progeny plants of step (c), that comprise said first, second and third transgenic events.

2. The method of claim 1, wherein:
i) the first donor/recurrent plant is generated by,
e) crossing a first donor plant with a recurrent plant to generate first progeny, wherein said first donor plant comprises said first transgenic event and said second transgenic event;
f) providing one or more backcross generations by crossing the first progeny of step (e), or optionally selfed offspring of the first progeny of step (e), with said recurrent plants to provide first backcross plants, and crossing said first backcross plants with said recurrent plants to produce said first donor/recurrent plant comprising said first transgenic event, said second transgenic event and having a recurrent parent percentage of greater than 80; and,
ii) the second donor/recurrent plant is generated by,
g) crossing a second donor plant with said recurrent plant to generate second progeny, wherein said second donor plant comprises said second transgenic event and said third transgenic event;
h) providing one or more backcross generations by crossing the second progeny of step (g), or optionally selfed offspring of the second progeny of step (g), with said recurrent plants to provide second backcross plants, and crossing said second backcross plants with said recurrent plants to produce said second donor/recurrent plant comprising said second transgenic event, said third transgenic event and having a recurrent parent percentage of greater than 80%.

3. The method of claim 2, wherein the recurrent parent plant is a female breeding plant in at least one of steps (e) through (h).

4. The method of claim 3 wherein the first and second progeny plants are each backcrossed with said recurrent plant at least twice to generate parallel lines of BC2 or higher first backcross progeny plants, representing the first donor/recurrent plant and second donor/recurrent plant, respectively.

5. The method of claim 2, wherein at least one of said first or second transgenic events of the first donor/recurrent plant is fixed as a homozygous trait.

6. The method of claim 2, wherein at least one of said second or third transgenic events of the second donor/recurrent plant is fixed as a homozygous trait.

7. The method of claim 1, wherein the first, second and third transgenic events are fixed as homozygous traits in the progeny plants comprising said first, second and third transgenic events.

8. The method of claim 1, wherein the progeny plants comprising said first, second and third transgenic events have a recurrent parent percentage of greater than 94%.

9. The method of claims 1, wherein the progeny plants comprising said first, second and third transgenic events have a recurrent parent percentage of at least 97.5%.

10. The method of claim 9, wherein the plant is a monocot plant.

11. The method of claim 10, wherein the monocot plant is selected from the group consisting of a corn plant, a wheat plant, a grass plant, and a rice plant.

12. The method of claim 9, wherein the plant is a dicot plant.

13. The method of claim 12, wherein the dicot plant is selected from the group consisting of a soybean plant, a canola plant, a tobacco plant, a tomato plant, a rapeseed plant, a Brassica plant, an alfalfa plant, a sugar beet plant, and a cotton plant.

14. The method of claim 1, wherein at least one of said first, second and third transgenic events comprises a transgene selected from the group consisting of an insecticidal resistance transgene, herbicide tolerance transgene, nitrogen use efficiency transgene, water use efficiency transgene, nutritional quality transgene, DNA binding transgene, and selectable marker transgene.

15. The method of claim 1 wherein one of said first and second donor/recurrent plant further comprises a fourth transgenic event.

16. The method of claim 15 wherein one of said first or second donor/recurrent plant further comprises a fifth transgenic event.

17. A method for introgressing three or more transgenic events into a plant, said method comprising
a) providing a first donor plant, comprising a first stack of at least two transgenic events;
b) crossing the first donor plant with a selected recurrent parent plant to produce a first F1 progeny plant that comprises said first stack of transgenic events;
c) performing a first breeding backcross of the first F1 progeny plant with the recurrent parent plant, and selecting a first breeding backcross progeny plant comprising said first stack of transgenic events
d) backcrossing the selected first breeding backcross progeny with the recurrent parent plant one or more times in succession to produce a BC2 or higher first backcross progeny plant comprising the first stack of transgenic events;
e) selecting a second donor plant comprising a second stack of at least two transgenic events, wherein at least one of the transgenic events of said second stack is also present in the first stack of transgenic events;
f) crossing the second donor plant and said selected recurrent parent plant to produce a second F1 progeny plant that comprises said second stack of transgenic events;
g) performing a second breeding backcross of the second F1 progeny plant to the recurrent parent plant, and selecting a second breeding backcross progeny plant comprising the second stack of transgenic events;
h) backcrossing the selected second breeding backcross progeny to the recurrent parent plant one or more times in succession to produce an BC2 or higher second backcross progeny plant comprising the second stack of transgenic events;
i) crossing the BC2 or higher first backcross progeny with the BC2 or higher second backcross progeny to produce a third progeny plant comprising three transgenic events from the first and second stacks of transgenic events.

18. The method of claim 17, wherein the third progeny plant comprises at least 97.5% of the recombinant parent genome.

19. The method of claim 17, wherein the recurrent parent plant is a female breeding plant in at least one of steps (c) through (h).

20. The method of claim 17, wherein the plant is a monocot plant.

21. The method of claim 20, wherein the monocot plant is selected from the group consisting of a corn plant, a wheat plant, a grass plant, and a rice plant.

22. The method of claim 17, wherein the plant is a dicot plant.

23. The method of claim 22, wherein the dicot plant is selected from the group consisting of a soybean plant, a canola plant, a tobacco plant, a tomato plant, a rapeseed plant, a Brassica plant, an alfalfa plant, a sugar beet plant, and a cotton plant.

24. The method of claim 17, wherein the transgenic event comprises at least one transgene selected from the group consisting of group consisting of an insecticidal resistance transgene, herbicide tolerance transgene, nitrogen use efficiency transgene, water use efficiency transgene, nutritional quality transgene, DNA binding transgene, and selectable marker transgene.

25. The method of any one of claims 17, wherein at least one of the transgenic events of the first stack or second stack is fixed as a homozygous trait in the first backcross progeny plant or second backcross progeny plant, respectively.

26. The method of any one of claims 17, wherein the third progeny plant comprises all transgenic events fixed as homozygous traits.

27. The method of claim 17, wherein the third progeny plant comprises less than 20 cM linkage drag from the first donor plant and/or second donor plant.

28. The method of claim 17, wherein the first backcross progeny plant of step (e) and the second backcross progeny plant of step (i) are selected via marker assisted selection, selected from the group consisting of SNP marker assisted selection, SSR marker assisted selection, RFLP marker assisted selection, Next Generation Sequencing assisted selection, RAPD marker assisted selection, and AFLP marker assisted selection.

29. A plant produced by the method of claim 17, wherein the plant comprises the first and second stack of transgenic events.
